(19) United States
(12) Patent Application Publication
(10) Pub. No.: US 2010/0292150 A1
(43) Pub. Date: Nov. 18, 2010

(54) Treatment of Menorrhagia with Aromatase Inhibitor

(75) Inventor: James Symons, Ann Arbor, MI (US)

Correspondence Address: Jonathan P. O''Brien, Ph.D.;Honigman Miller Schwartz and Cohn, 350 East Michigan Avenue, Suite 300, KALAMAZOO, MI, 49007, US

(73) Assignee: MEDITRINA PHARMACEUTICALS, INC., Ann Arbor, MI (US)

(21) Appl. No.: 12/746,965

(22) PCT Filed: Dec. 10, 2008
(86) PCT No.: PCT/US2008/013545

§ 371 Date: Jun. 16, 2010

Related U.S. Application Data
(60) Provisional application No. 61/012,686, filed on Dec. 10, 2007.

(51) Int. Cl. A61K 31/4196; A61K 31/5685; A61K 31/58; A61K 38/23; A61P 7/00; A61P 19/00; A61P 15/00; A61P 7/04

(52) U.S. Cl. 514/11.9; 514/383; 514/177; 514/176

(57) ABSTRACT

The present invention provides a method for treating menorrhagia in a premenopausal woman, which includes administering to the woman in need thereof a pharmaceutically effective amount of an aromatase inhibitor. Also within the scope of this invention is a pharmaceutical composition for the treatment.

### CROSS REFERENCE TO RELATED APPLICATIONS

This application claims priority to U.S. Ser. No. 61/012,686, filed Dec. 10, 2007. The entire content of the aforementioned application is incorporated herein.

### BACKGROUND OF THE INVENTION

A normal menstrual cycle is 21-35 days in duration, with bleeding lasting an average of 5 days and total blood flow between 25 and 80 mL. A blood loss of greater than 80 ml or lasting longer than 7 days constitutes menorrhagia (also called hypermenorrhea). In other words, menorrhagia is an abnormally heavy and prolonged menstrual period at regular intervals. It adversely affects the life quality of 9%-14% of otherwise healthy women. In some instances, due to excessive blood loss, it results in anemia, fatigue, and syncope.

It has been reported that menstrual disorders account for 19% of the 20 million physician office visits over two years (see, e.g., Albers, J. R. et al., Am Fam Physician., 2004, 15; 69(8):1915-26; Nicholson, W. K. et al., Am J Obstet Gynecol., 2001, 184:523-30). In addition, abnormal uterine bleeding is reportedly involved in 25% of gynecologic surgeries (see, e.g., Goodman, A., Clin Cornerstone, 2000, 3:25-35) and almost 30% of hysterectomies that take place in the United States are performed to alleviate heavy menstrual bleeding (see, e.g., Shaw, J. A. et al., Menorrhagia, eMedicine, 2007, http://www.emedicine.com/med/topic1449.htm). While hysterectomy or less invasive hysteroscopic procedures such as endometrial ablation provide definitive correction of menorrhagia, current gynecologic practices tend toward more conservative therapy that is less expensive, uses fewer health care resources, and, most importantly for premenopausal women, preserves ovarian function for childbearing, health, and quality of life considerations. Another telling statistic is that nearly 50% of uterine pathology findings from hysterectomies for menorrhagia are free of disease and histopathologic abnormalities (see, e.g., Shaw, J. A. et al., Menorrhagia, eMedicine, 2007, http://www.emedicine.com/med/topic1449.htm).

Given this, there continues to be a need to increase the number and effectiveness of approved medical treatments for menorrhagia, especially for premenopausal women.

The underlying causes of menorrhagia are varied and can be difficult to determine. For the majority of women experiencing menorrhagia, the cause may be related to abnormalities in hormonal production resulting in an imbalance between estrogen and progesterone secretion. Other causes of menorrhagia can be organic or structural, such as fibroids, polyps, cysts, endometrial carcinoma, complications of pregnancy, or methods of contraception (see, e.g., Ely, J. W. et al., J Am Board Fam Med., 2006, 19:590-602). Therefore, most often treatment focuses on normalizing the production of hormones involved in the menstrual cycle.

For premenopausal women with menorrhagia, first line treatment is generally oral contraceptives. If oral contraceptives are contraindicated (e.g., due to a history of thrombosis, stroke, or smoking), oral progestin or progesterone-releasing intrauterine systems are alternative treatment options. Duration of treatment is generally three or more menstrual cycles (see, e.g., Ely, J. W. et al., J Am Board Fam Med., 2006, 19:590-602; Lethaby, A. et al., Cochran Data Base Syst. Rev., 2005 (4), CD002126).

If a nonhormonal treatment option is called for, non steroidal anti-inflammatory drugs (NSAIDS) shown to reduce blood loss can be prescribed (see, e.g., Lethaby, A. et al., Cochran Data Base Syst. Rev., 2002 (1):CD000400; Dawood, M. Y., Am J Obstet Gynecol., 1993, 169: 1255-65).

Other treatment options, depending on the suspected or known underlying cause of menorrhagia, include prostaglandin inhibitors, gonadotropin-releasing hormone agonists, danazol, and antifibrinolytics (see, e.g., Wellington, K. et al., Drugs, 2003, 63:1417-33; Lethaby, A. et al., Cochran Data Base Syst. Rev., 2000 (2):CD000249).

The above-mentioned treatments have their respective disadvantages. For instance, it was reported that treatments with nonsteroidal anti-inflammatory drugs (NSAIDs), oral progestins (e.g., norethisterone or norethindrone), or oral contraceptives were often ineffective and even caused many women to undergo surgical procedures such as endometrial resection or ablation or hysterectomy. See, e.g., Nagrani, R. et al., Br. J. Obstet. Gynaecol., 2002; 109:345-347. Additionally, treatment with contraceptive showed poor cycle control and sometime significant breakthrough bleeding was still observed after the treatment. Thus, an alternative treatment with consistent or better efficacy and less side effects is desired.

### SUMMARY OF THE INVENTION

In one aspect, the present invention provides a method for treating menorrhagia in a premenopausal woman, which includes administering to the woman in need thereof a pharmaceutically effective amount of an aromatase inhibitor. As used herein, the term “premenopausal woman” encompasses both a premenopausal woman and a perimenopausal woman.

In some embodiments, the administration of the aromatase inhibitor starts on the second day of a menstrual cycle. In some other embodiments, the aromatase inhibitor is administered once a day. In some embodiments, the aromatase inhibitor is administered daily for about 6 to about 26 days (e.g., from about 20 to about 26 days, or for about 26 days) in each menstrual cycle.

In some embodiments, the amount of the aromatase inhibitor administered daily can range from about 1 mg to about 240 mg (e.g., from about 2.5 mg to about 60 mg, or from about 10 mg to about 30 mg).

In some embodiments, the aromatase inhibitor is anastrozole, letrozole, exemestane, vorozole, formestane, or 1,4,6-androstatrien-3,17-dione. In some further embodiments, the aromatase inhibitor is anastrozole, letrozole, or exemestane.

In some other embodiments, letrozole in the amount of about 2.5 mg to about 60 mg (e.g., from about 2.5 mg to about 30 mg, or from about 2.5 mg to about 15 mg) is administered daily. In some other embodiments, anastrozole in the amount of about 1 mg to about 60 mg is administered daily. In still some other embodiments, exemestane in the amount of about 25 mg to about 200 mg is administered daily.

In some embodiments, about 20 mg of an aromatase inhibitor (e.g., anastrozole or letrozole) is administered on the first day of the treatment. In some other embodiments, about 10 mg of an aromatase inhibitor (e.g., anastrozole or letrozole) is administered on and after the second day of the treatment.

In some embodiments, the aromatase inhibitor is administered during the luteal phase (i.e., menstrual cycle days 15 to 21) of a menstrual cycle.

In some embodiments, the aromatase inhibitor is administered orally.

In some embodiments, the method of this invention further includes administration of an additional therapeutic agent which does not interfere with the efficacy of the aromatase inhibitor in treating menorrhagia. Examples of such an additional therapeutic agent include an oral contraceptive, a progestin, progesterone, a testosterone agonist, nonsteroidal anti-inflammatory drugs (NSAIDS), a prostaglandin inhibitor, a gonadotropin-releasing hormone agonist, an antifibrinolytic agent, or a bone anti-absorptive. An example of an oral contraceptive is a combination of estrogen and a progestin. Examples of progestins are norethynodrel, norethindrone, norgestimate, norgestrel, levonorgestrel, medroxyprogesterone, or desogestrel. An example of a testosterone agonist is danazol. Examples of NSAIDS are aspirin, ibuprofen, naproxen, nabumetone, or other inhibitors of cyclooxygenase (COX). Examples of antifibrinolytic agents are e-aminocaproic acid, tranexamic acid, or aprotinin. An example of a bone anti-absorptive is calcitonin.

In some embodiments, the method of the invention includes the administration of an aromatase inhibitor, without an additional therapeutic agent, in treating menorrhagia. Examples of such an additional therapeutic agent include an oral contraceptive, a progestin or progesterone, a testosterone agonist, nonsteroidal anti-inflammatory drugs (NSAIDS), a prostaglandin inhibitor, a gonadotropin-releasing hormone agonist, an antifibrinolytic agent, or a bone anti-absorptive.

In some embodiments, the method of this invention includes a pharmaceutical composition for treating menorrhagia in a premenopausal woman, comprising an aromatase inhibitor and a pharmaceutically acceptable carrier, wherein the pharmaceutical composition is administered to the woman daily to provide the aromatase inhibitor in a pharmaceutically or therapeutically effective amount, e.g., from about 1 mg to about 240 mg.

In some embodiments, the aromatase inhibitor contained in the pharmaceutical composition of this invention can be anastrozole, letrozole, or exemestane. In some other embodiments, the composition can further include an oral contraceptive, a progestin, a progesterone, a testosterone agonist, a NSAIDS, a prostaglandin inhibitor, a gonadotropin-releasing hormone agonist, an antifibrinolytic agent, or a bone anti-absorptive.

A detailed description of some of the features of the present invention is provided below.

### BRIEF DESCRIPTION OF FIGURES

FIG. 1 shows the bleeding pattern of premenopausal women in the Control Group who each received a placebo.

FIG. 2 shows the bleeding pattern of premenopausal women in the Treatment Group who each received anastrozole.

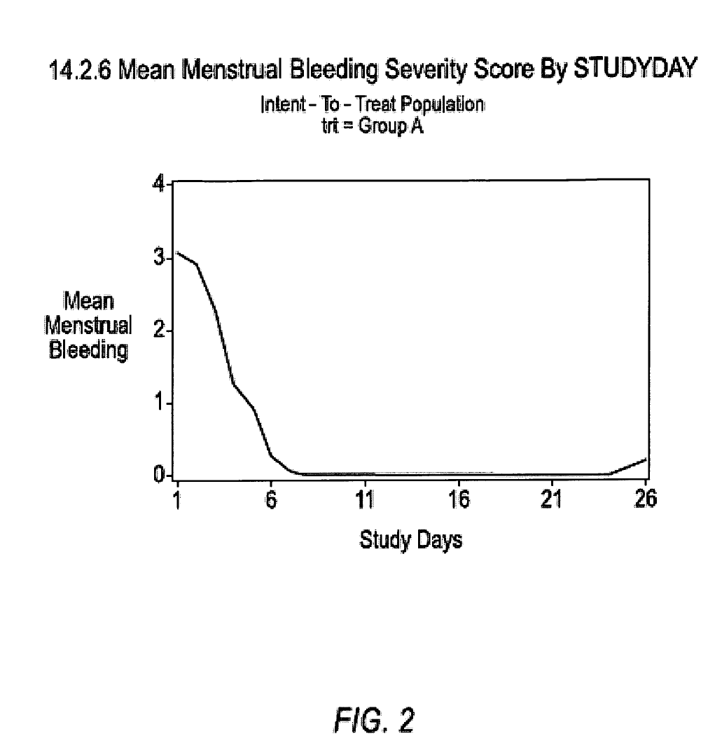

### DETAILED DESCRIPTION OF THE INVENTION

In one aspect, the present invention provides a method for treating menorrhagia in a premenopausal woman, which includes administering to the woman in need thereof an aromatase inhibitor, e.g., daily for a period of 5 to 26 days.

Aromatase inhibitors are compounds that have inhibitory effect on the enzyme aromatase which is responsible for aromatizing androgens (that is, to selectively increase their aromaticity) to produce estrogens. A number of such compounds have been used in the treatment of breast cancer in postmenopausal women. Examples of aromatase inhibitors have been extensively reported in scientific or technical literature. See, e.g., WO 2006/041941 and WO 2006/041939, the contents of which are incorporated herein by reference in their entireties Examples of currently available aromatase inhibitor drugs include aminoglutethimide, anastrozole (marketed under the tradename Arimidex®), letrozole (marketed under the tradename Femara®), and exemestane (marketed under the tradename Aromasin®). Additionally, 4-androstene-3,6,17-trione (“6-OXO”) is also an aromatase inhibitor, marketed as a nutritional supplement for athletes and weight lifters). Additional examples of aromatase inhibitors are described in British Patent Application No. GB-A-2 17 1100, European Patent Publication Nos. EP-A 165 904, EP-A 281 283, EP-A 296 749, EP-A 299 684, EP-A 236 940, EP-A 408 509, EP-A 316 097, EP-A 354 689, EP-A 354 683, EP-A 181 287, EP-A 337 928, EP-A 340 153, EP-A 114 033, EP-A 166 692, and EP-A 337 929; Swiss Patent Application Nos. 1339/90-7, 3014/90-0, 3014/90-0, and 3923/90-4; German Patent Application Nos. DE-A 4 014 006, DE-A 3 926 365, and DE-A 3 740 125; U.S. Pat. No. 4,322,416, and U.S. Provisional Application Ser. No. 60/615,978. All of the publications cited herein are incorporated herein by reference in their entireties.

The aromatase inhibitors that can be used for the method of this invention generally have in vitro inhibition activities exhibiting IC₅₀ values of 10⁻²M or lower, e.g., 10⁻³ M or lower, 10⁻⁴M or lower, 10⁻⁵ M or lower, 10⁻⁶M or lower, or 10⁻⁷ M or lower. The in vitro inhibition of aromatase activity of a substance can be demonstrated, for example, by using the methods described in J. Biol. Chem., 1974, 249: 5364, or in J. Enzyme Inhib., 1990, 4: 169. In addition, the IC₅₀ values for aromatase inhibition can be obtained, for example, in vitro by a direct product isolation method relating to inhibition of the conversion of androstenedione to oestrone in human placental microsomes.

The in vivo aromatase inhibition activity of a substance can be determined, for example, by the method as described in J. Enzyme Inhib., 1990, 4: 79. Specifically, androstenedione (30 mg/kg subcutaneously) is administered on its own or together with an aromatase inhibitor (orally or subcutaneously) to sexually immature female rats for a period of 4 days. After the fourth administration, the rats are sacrificed and the uteri are isolated and weighed. The aromatase inhibition is determined by the extent to which the hypertrophy of the uterus induced by the administration of androstenedione alone is suppressed or reduced by the simultaneous administration of the aromatase inhibitor.

As used herein, the term “menstrual cycle” refers to a recurring cycle of physiological changes in the females of some animal species that is associated with reproductive fertility. While the cycle length may vary from woman to woman, 28 days is generally taken as representative of the average ovulatory cycle in women. For the purpose of this invention, the onset of menstrual bleeding marks the beginning of the cycle, so the first day of bleeding is also referred to as the first day of the cycle.

In this method, the amount of aromatase inhibitor can vary according to the specific need, the specific type of aromatase inhibitor, and the woman's physical or medical conditions. The appropriate amount of aromatase inhibitor in each dosage can also be determined by a doctor in view of the overall physical condition of the woman. Generally, a therapeutically or pharmaceutically “effective amount” is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, New York, 537 (1970). As used herein, a “patient” refers to a premenopausal or perimenopausal woman who is characteristic of symptoms of menorrhagia. Examples of the therapeutically effective amount of an aromatase inhibitor for the method of this invention can range from about 1 mg to about 240 mg, depending on the specific type of the aromatase inhibitor.

The method of this invention can be carried out by administering a pharmaceutical composition of this invention which includes an aromatase inhibitor and a pharmaceutically acceptable carrier, adjuvant, vehicle, or excipient, wherein the pharmaceutical composition is administered to the woman daily to provide the aromatase inhibitor in a pharmaceutically or therapeutically effective amount, e.g., from about 1 mg to about 240 mg.

Aromatase inhibitors can be used to practice the present invention in the same way as most other types of compounds for their respective pharmaceutical applications. They can be formulated, together with a pharmaceutically acceptable carrier, adjuvant, vehicle, or excipient, into pharmaceutical compositions for various routes of administration, e.g., for enteral, such as peroral or rectal administration, also for transdermal or sublingual administration, and for parenteral, for example intravenous, subcutaneous and intramuscular, administration.

The proportion of active ingredient in such pharmaceutical compositions is generally from approximately 0.001% to approximately 60%, e.g., from approximately 0.1% to approximately 20%.

Suitable excipients for pharmaceutical compositions for oral administration are, e.g., fillers, such as sugars, for example lactose, saccharose, mannitol or sorbitol, cellulose preparations and/or calcium phosphates, for example tricalcium phosphate or calcium hydrogen phosphate, and binders, such as starches, for example corn, wheat, rice or potato starch, gelatin, tragacanth, methylcellulose and/or hydroxypropylcellulose, disintegrators, such as the above-mentioned starches, also carboxymethyl starch, crosslinked polyvinylpyrrolidone, agar, alginic acid or a salt thereof, such as sodium alginate, and/or cellulose, for example in the form of crystals, especially in the form of microcrystals, and/or flow regulators and lubricants, for example silicic acid, talc, stearic acid or salts thereof, such as magnesium or calcium stearate, cellulose and/or polyethylene glycol.

Examples of orally administrable pharmaceutical compositions are dry-filled capsules consisting of gelatin, and also soft sealed capsules consisting of gelatin and a plasticiser, such as glycerol or sorbitol. The dry-filled capsules may contain the active ingredient in the form of granules, for example in admixture with fillers, such as lactose, binders, such as starches, and/or glidants, such as talc or magnesium stearate, and, if desired, stabilisers. In soft capsules, the active ingredient is preferably dissolved or suspended in suitable oily excipients, such as fatty oils, paraffin oil or liquid polyethylene glycols, to which stabilisers and/or anti-bacterial agents may also be added. Capsules may also be used. These are easily bitten through and have the advantage of rapid sublingual absorption of the active ingredient.

The pharmaceutical compositions that can be used for the present invention can be prepared in a known manner, e.g., by means of conventional mixing, granulating, confectioning, dissolving, coating or lyophilizing processes. For example, pharmaceutical compositions for oral administration can be obtained by combining the active ingredient with solid carriers, optionally granulating a resulting mixture, and processing the mixture or granules, if desired or necessary after the addition of suitable excipients, to form tablets, beads, pellets, or cores.

### Example

Women with menorrhagia were enrolled in the study and divided into two groups—one as Treatment Group and the other as Control Group. Administering of anastrozole to the women in the Treatment Group started on the second day of the menstrual cycle and lasted for 26 days. In some cases, the administration started on the third day due to site scheduling limitations. Conversely, the women in the Control Group received placebo during the same period. Data on the mean menstrual bleeding severity score were recorded on a daily basis according to the following categories of vaginal bleeding: 0=none; 1=spotting; 2=mild bleeding; 3=moderate bleeding; and 4=heavy bleeding.

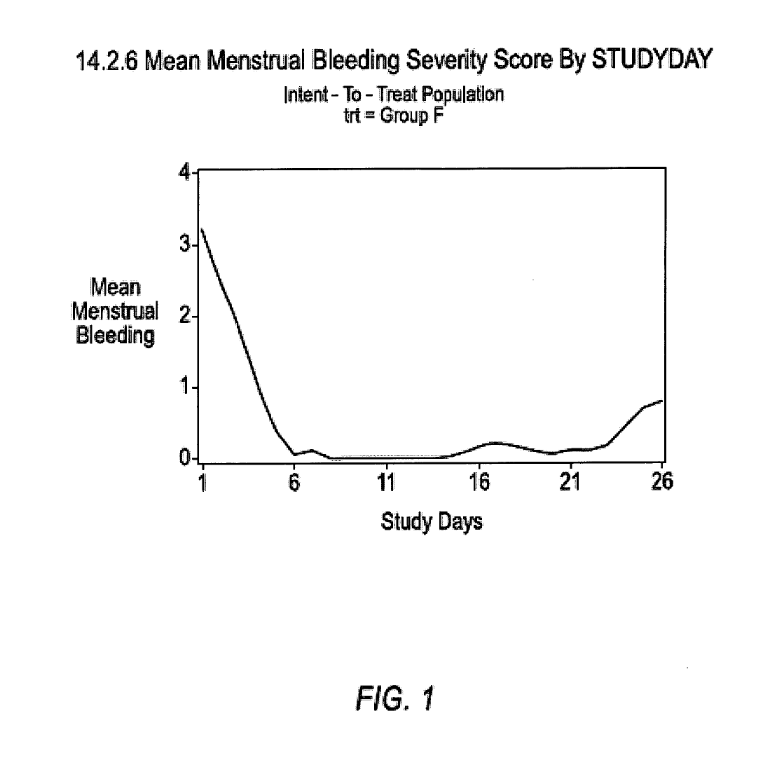

In the Control Group, within 6 days, the average score was down to about zero. Over the next 20 days, the fluctuations in menstrual bleeding severity would also be considered typical in that some women experience “breakthrough bleeding” which was reflected in the deviation from zero that would be expected in models of normal cycles, but was not reflected in reality. This was particularly notable during the second half of the menstrual cycle. The data are shown in FIG. 1.

The observation above was contrasted by women who received anastrozole for 26 consecutive days (Treatment Group). On Study Day 1, which corresponds to Cycle Day 2 as with the Control Group, the women were administered 20 mg of anastrozole in the clinic office and then 10 mg/day for the remaining 25 days. While there was comparable mean bleeding severity at the start of the treatment interval (˜3), there was a notable difference in reported bleeding with active treatment over the remaining days in that there was no bleeding or spotting reported until the very end of the cycle. The data are shown in FIG. B. This unexpected contrast in patterns strongly suggests that anastrozole treatment in a premenopausal population of women provided cycle control in contrast to a group that has self-reported normal cycles prior to study entry. In a population of women that has significant menstrual bleeding abnormalities, most notably menorrhagia, this provided a therapeutic option that does not appear to have any significant safety liabilities, given that the aromatase inhibitor generally exhibited a safety profile as compared to placebo.

Three aromatase inhibitors marketed in the US for long-term chronic breast cancer therapy (five years and beyond)—anastrozole, letrozole, and exemestane—show that they are generally accepted as safe drugs. Studies of AI use in premenopausal women in fertility clinics report that the drugs were well tolerated (see, e.g., Al-Fadhli R. et al., Fertil Steril., 2006, 85:161-164; Al-Fozan, H. et al., Fertil Steril., 2004, 82: 1561-1563; Bayar, U. et al., Fertil Steril., 2006, 85:1045-1048; Cortinez A. et al., Fertil Steril., 2005, 83:110-115; Garcia-Velasco, J. A. et al., Fertil Steril., 2005, 84:82-87; Healey, S. et al., Fertil. Steril., 2003, 80:1325-1329; Holzer, H. et al., Fertil Steril., 2006, 85:277-284; Jee, B. C. et al., Fertil Steril., 2006, 85: 1774-1777). Specific to anastrozole, in a phase 1 single and multiple dose study of anastrozole (5, 10, 15, and 20 mg) in 26 healthy premenopausal women there were no severe, serious, or otherwise significant adverse events (see, e.g., Tredway, D. R. et al., Fertil Steril., 2004, 82:1587-93).

All three aromatase inhibitors should be applicable for the method of this invention.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of this invention.

What is claimed is:

1. A method for treating menorrhagia in a premenopausal woman, comprising administering to the woman in need thereof a pharmaceutically effective amount of an aromatase inhibitor.

2. The method of claim 1, wherein the administration of the aromatase inhibitor starts on the second day of a menstrual cycle.

3. The method of claim 1, wherein the aromatase inhibitor is administered once a day.

4. The method of claim 1, wherein the aromatase inhibitor is administered daily for about 6 to about 26 days in each menstrual cycle.

5. (canceled)

6. (canceled)

7. The method of claim 1, wherein the aromatase inhibitor is administered daily in the amount of about 1 mg to about 240 mg.

8. (canceled)

9. (canceled)

10. The method of claim 1, wherein the aromatase inhibitor is anastrozole, letrozole, exemestane, vorozole, formestane, or 1,4,6-androstatrien-3,17-dione.

11. (canceled)

12. The method of claim 10, wherein about 2.5 mg to about 30 mg of letrozole is administered daily.

13. The method of claim 10, wherein about 1 mg to about 60 mg of anastrozole is administered daily.

14. (canceled)

15. (canceled)

16. The method of claim 10, wherein about 25 mg to about 200 mg of exemestane is administered daily.

17. The method of claim 1, wherein the aromatase inhibitor is administered during the luteal phase of a menstrual cycle.

18. The method of claim 1, wherein the aromatase inhibitor is administered orally.

19. The method of claim 1, further comprising administering to the woman an oral contraceptive.

20. The method of claim 1, further comprising administering to the woman a progestin or progesterone, or testosterone agonist.

21. The method of claim 20, wherein the testosterone agonist is danazol.

22. The method of claim 20, wherein the progestin is norethynodrel, norethindrone, norgestimate, norgestrel, levonorgestrel, medroxyprogesterone, or desogestrel.

23. The method of claim 1, further comprising administering to the woman a NSAIDS.

24. The method of claim 1, further comprising administering to the woman a prostaglandin inhibitor.

25. The method of claim 1, further comprising administering to the woman a gonadotropin-releasing hormone agonist.

26. The method of claim 1, further comprising administering to the woman an antifibrinolytic agent.

27. The method of claim 26, wherein the antifibrinolytic agent is e-aminocaproic acid, tranexamic acid, or aprotinin.

28. The method of claim 1, further comprising administering to the woman a bone anti-absorptive.

29. The method of claim 28, wherein the bone anti-absorptive is calcitonin.

30. A method for treating menorrhagia in a premenopausal woman, consisting essentially of administering to the woman in need thereof a pharmaceutically effective amount of an aromatase inhibitor.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. The method of claim 30, wherein the aromatase inhibitor is anastrozole, and about 1 mg to about 60 mg of anastrozole is administered daily.

43. The method of claim 42, wherein 20 mg of aromatase inhibitor is administered on the first day of the treatment.

44. The method of claim 42, wherein about 10 mg of aromatase inhibitor is administered on and after the second day of the treatment.

45. (canceled)

46. The method of claim 30, wherein the aromatase inhibitor is administered during the luteal phase of a menstrual cycle.

47. (canceled)

48. A pharmaceutical composition for treating menorrhagia in a premenopausal woman, comprising an aromatase inhibitor and a pharmaceutically acceptable carrier, wherein the pharmaceutical composition is administered to the woman daily to provide the aromatase inhibitor in the amount of about 1 mg to about 240 mg.

49. A pharmaceutical composition for treating menorrhagia in a premenopausal woman, consisting essentially of an aromatase inhibitor and a pharmaceutically acceptable carrier, wherein the pharmaceutical composition is administered to the woman daily to provide the aromatase inhibitor in the amount of about 1 mg to about 240 mg.

50. The composition of claim 48, further comprising an oral contraceptive, a progestin, progesterone, a testosterone agonist, a NSAIDS, a prostaglandin inhibitor, a gonadotropin-releasing hormone agonist, an antifibrinolytic agent, or a bone anti-absorptive.
